(19) United States
(12) Patent Application Publication
(10) Pub. No.: US 2012/0178101 A1
(43) Pub. Date: Jul. 12, 2012

(54) DEVICE, METHOD, AND SYSTEM FOR QUANTITATIVELY MEASURING A SPECIMEN USING A CAMERA

(75) Inventors: Byeong-Woo Bae, Anyang-si (KR); Cheol-Min Park, Anyang-si (KR)

(73) Assignee: Infopia Co., Ltd., Anyang-si, Gyeonggi-do (KR)

(21) Appl. No.: 13/393,989

(22) PCT Filed: Aug. 30, 2010
(86) PCT No.: PCT/KR2010/005823

§ 371 Date: Apr. 2, 2012

(30) Foreign Application Priority Data
Sep. 3, 2009 (KR) 10-2009-0082869

(51) Int. Cl. G01N 33/573; C12M 1/34; G01N 33/566; G01N 30/00

(52) U.S. Cl. 435/7.4; 422/69; 435/287.2; 436/501; 422/430

(57) ABSTRACT

The present invention relates to a device and method for quantitatively measuring an analyte using a camera. More particularly, to capture the identification code required for obtaining an accurate analysis result for the analyte to be analyzed, to capture the result of the reaction of the analyte using a camera without additional equipment, and to read the identification code and the result of the reaction of the analyte, the device of the present invention comprises: a camera for capturing a camera recognition area of an analyte kit, which contains an analyte reaction result obtained by the reaction of the analyte and an identification code for the analyte kit; an image-processing unit for separating the analyte reaction result image and the identification code image from images of the camera recognition area of the analyte kit captured by the camera; a reading unit for reading the analyte reaction result image and the identification code image; and a control unit for enabling the read result of the analyte reaction result image to be processed using the read result of the identification code image.

### TECHNICAL FIELD

The present invention relates to a device, method, and system for quantitatively measuring an analyte, and more particularly, to a device, method, and system for quantitatively measuring an analyte by using a camera, which can read an identification code required to derive an accurate analysis result of an analyte while photographing an analyte reaction result by using a camera without additional equipment.

### BACKGROUND ART

In general, as interest in health increases, various technologies for physical health analysis using bio-samples are being introduced.

From thereamong, using diagnostic kits allows an extremely small amount of bio-analytes to be analyzed, thereby easily checking health conditions.

In this case, in order to analyze an end result as illustrated in FIG. 1, a result of an antigen-antibody reaction or enzyme reaction occurring in samples is provided through a diagnostic kit 10, and the result is photographed by a camera 20.

At this point, to obtain an accurate result, code information including effective information about respective diagnostic kits should be recognized.

To recognize this code information, according to existing methods, codes are implemented in bar codes, and the code information is recognized using a barcode reader separately from a camera.

Accordingly, as illustrated in FIG. 2, a reaction result is photographed using a camera 220, and a code 230 is read using a bar code reader 240 separately. In another method, codes may be recognized using RFID units.

However, using typical bar code readers or RFID units needs additional equipment for reading codes separately from photographing a reaction result of a diagnostic kit, and thus the miniaturizing of an analyzer is difficult and also additional cost arises.

Moreover, since additional operations of reading codes are needed, it takes more time for analysis and also operation processes are more complicated.

### DISCLOSURE OF THE INVENTION

### Technical Problem

The present invention provides a quantitative measurement device using a camera, which can obtain code information about an analyte kit as well as a sample reaction result through the photographing of images when analyzing the sample reaction result.

The present invention also provides an analyte kit, which can have an image photographable area to analyze the sample reaction result and the code information of the analyte kit through the photographing of images.

### Technical Solution

A quantitative measurement device using a camera according to a first embodiment of the present invention includes a camera photographing a camera recognition area in one side of an analyte kit, wherein the camera recognition area comprises an analyte reaction result obtained by reaction of the analyte in blood and an identification code of the analyte kit; an image processing unit separating the images of the analyte reaction result and identification code from an image of the camera recognition code of the analyte kit photographed by the camera; a read unit reading the images of the photographed analyte reaction result and identification code, and a control unit allowing a result of reading the image of the analyte reaction result to be processed by using a result of reading the image of the identification code.

In this case, the read unit may include a first read unit reading the image of the analyte reaction result and a second read unit reading the image of the identification code.

And also, in order to process the result of reading the image of the analyte reaction result, the quantitative measurement device may further comprise a storage unit storing data corresponding to the identification code.

The result of reading the image of the analyte reaction result may be output through an output unit or display unit.

A quantitative measurement device using a camera according to a second embodiment of the present invention includes a camera photographing a camera recognition area in one side of an analyte kit in the order of a first image and a second image, wherein the camera recognition area comprises an analyte reaction result obtained by reaction of the analyte in blood and an identification code of the analyte kit; a read unit reading the analyte reaction result and the identification code from the first image and the second image, respectively, which are photographed by the camera; and a control unit allowing a result of reading the image of the analyte reaction result to be processed by using a result of reading the image of the identification code.

The read unit extracts and reads the analyte reaction result and the identification code from a first virtual area and a second virtual area, respectively, of the camera recognition area in one side of the analyte kit.

The second virtual area may be a remaining area in the second image photographed by the camera, except the first virtual area.

The identification code may include at least one of a bar code, color pattern code, character, or number.

The identification code may include manufacturing lot information of the analyte kit, and the control unit may allow the result of reading the image of the analyte reaction result to be calibrated using the manufacturing lot information and allow the calibrated result to be output through a display unit.

The identification code may include an expire date of the analyte kit. In this case, the camera photographs the identification code before the analyte of blood is injected, and the control unit allows an error message to be output if the expire date of the analyte kit has passed.

The identification code may include analyte type information about types of analytes capable of being analyzed in the analyte kit. In this case, the camera photographs the identification code before the analyte of blood is injected, and the control unit allows an error message to be output if the analyte has the type which cannot be analyzed in the analyte kit.

An analyte kit used in a quantitative measurement device for quantitatively measuring an analyte in blood by using a camera according to a third embodiment of the present invention includes a display window displaying an analyte reaction result obtained by reaction of the analyte in blood; and an identification code of the analyte kit, wherein the display window and the identification code are formed in a camera recognition area in one side of the analyte kit.

The analyte may be an antigen, and the analyte reaction result may be a result of an antigen-antibody reaction. The antigen may be Myoglobin, Creatine Kinase-MB(CK-MB), Troponin I, Pro-BNP, or D-dimer.

The antigen may be a tumor marker. For example, the tumor marker may be CA15-3 for metastatic breast cancer detection, CA19-9 for rectal cancer and pancreatic cancer detection, CA125 for ovarian cancer, CEA for rectal cancer, lung cancer, pancreatic cancer, gastrointestinal cancer, and breast cancer detection, PAS for prostate cancer detection, or AFP for liver cancer detection.

The camera recognition area may include a first virtual area and a second virtual area, the first virtual area including the display window and the second virtual area including the identification code.

The first virtual area and the second virtual area may be separated with a visible line.

A quantitative measurement method using a camera according to a fourth embodiment of the present invention includes the steps of: photographing a camera recognition area in one side of an analyte kit by the camera, wherein the camera recognition area comprises an analyte reaction result obtained by reaction of the analyte in blood and an identification code of the analyte kit; separating the images of the analyte reaction result and identification code from an image of the camera recognition code of the analyte kit photographed by the camera; reading the images of the analyte reaction result and identification code, and processing a result of reading the image of the analyte reaction result by using a result of reading the image of the identification code.

The processing may use the data corresponding to the identification code stored in the storage unit to process the result of reading the analyte reaction result.

A quantitative measurement method using a camera according to a fifth embodiment of the present invention includes the first step of photographing a camera recognition area in one side of an analyte kit in the order of a first image and a second image by the camera, wherein the camera recognition area comprises an analyte reaction result obtained by reaction of the analyte in blood and an identification code of the analyte kit; the second step of reading the analyte reaction result from the first image; the third step of reading the identification code from the second image; and a fourth step of processing a result of reading the image of the analyte reaction result by using a result of reading the image of the identification code.

A quantitative measurement system using a camera according to a sixth embodiment of the present invention includes: an analyte kit including a display window displaying an analyte reaction result obtained by reaction of the analyte in blood, and an identification code included in a camera recognition area which is in the same side as the display window; and the quantitative measurement device according to the first or second embodiment.

### Advantageous Effects

As described above, the device and method for quantitatively measuring an analyte by using a camera according to the present invention can recognize an analyte reaction result and code information by using one camera, thereby analyzing a sample quickly and miniaturizing the device because there is no need to include a code recognition device separately.

Furthermore, the device and method for quantitatively measuring an analyte by using a camera according to the present invention can recognize an analyte reaction result and code information by using one camera, thereby analyzing the recognized information with software.

### BRIEF DESCRIPTION OF THE DRAWINGS

FIG. 1 is a view of a system for quantitatively measuring an analyte in the related art.

FIG. 2 is a view illustrating a concept of the prior art.

FIG. 3 is a view illustrating a basic concept of the present invention.

FIG. 4 is a view illustrating an analyte kit according to a first embodiment of the present invention.

FIG. 5 is a view illustrating various methods of implementing identification codes according to the present invention.

FIG. 6 is a view illustrating an analyte kit according to a second embodiment of the present invention.

FIG. 7 is a view illustrating a configuration of a system for quantitatively measuring an analyte by using a camera according to the present invention.

FIG. 8 is a view illustrating a method of quantitatively measuring an analyte by using a camera according to a first embodiment of the present invention.

FIG. 9 is a view illustrating a method of quantitatively measuring an analyte by using a camera according to a second embodiment of the present invention.

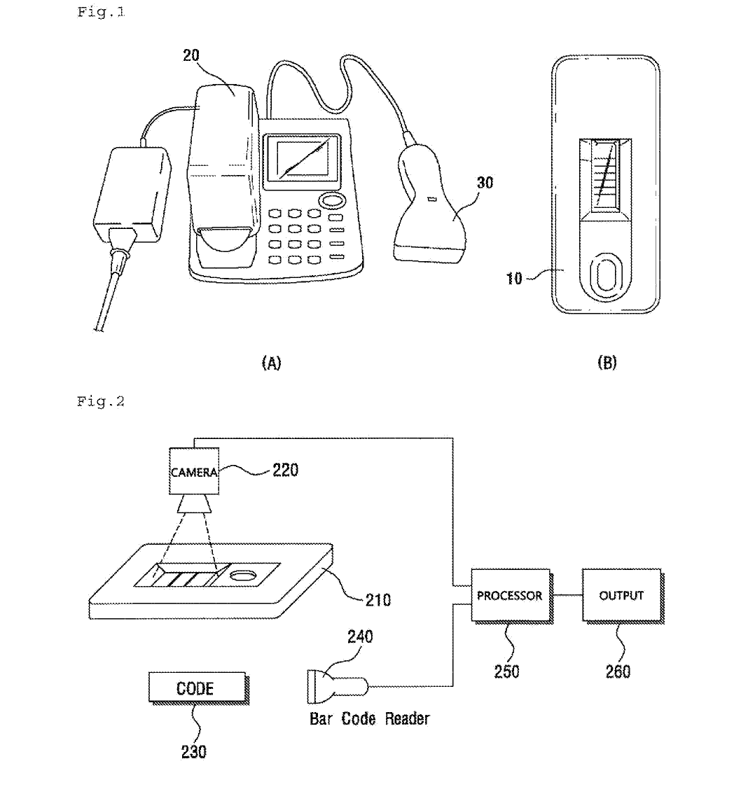

### BEST MODE FOR INVENTION

Hereinafter, preferred embodiments will be described in more detail with reference to the accompanying drawings. Moreover, detailed descriptions related to well-known functions or configurations will be ruled out in order not to unnecessarily obscure subject matters of the present invention.

In the description of the present invention, a “analyte” term is uniformly used as an analyte in a sample taken from an organism and then used for analysis, and a “analyte kit” term is uniformly used as a diagnostic kit. And also, an “identification code” term is uniformly used as a code including code information. However, technical features are not limited to these terms.

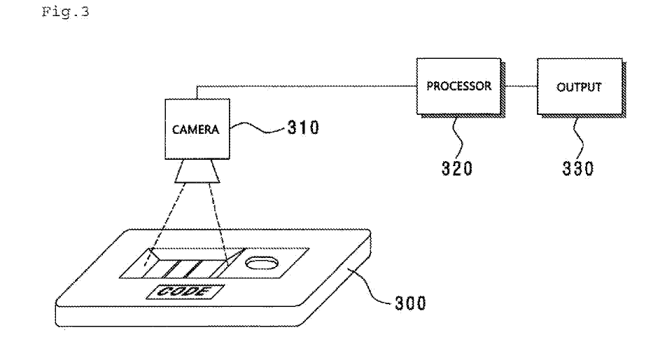

FIG. 3 is a view illustrating a basic concept of the present invention.

As illustrated in FIG. 3, in order to analyze an analyte through a quantitative measurement device according to the present invention, an analyte reaction result and an identification code are in the same side of an analyte kit 300.

At this point, an analyte obtained from blood is provided to the analyte kit 300, moved by chromatography into a reaction area of the analyte kit 300, and reacted therein. The result of the reaction area is referred to as the analyte reaction result.

For example, blood is moved by chromatography into a reaction area and reacted therein, and thus the color of the reaction area changes according to a concentration of an analyte in the blood. At this point, the color may be the analyte reaction result.

As a specific example, the analyte may be an antigen, and the analyte reaction result may be a result of an antigen-antibody reaction. Herein, the antigen may be Myoglobin, Creatine Kinase-MB(CK-MB), Troponin I, Pro-BNP, or D-dimer.

And also, the antigen may be a tumor marker. For example, the tumor marker may be CA15-3 for metastatic breast cancer detection, CA19-9 for rectal cancer and pancreatic cancer detection, CA125 for ovarian cancer, CEA for rectal cancer, lung cancer, pancreatic cancer, gastrointestinal cancer, and breast cancer detection, PAS for prostate cancer detection, or AFP for liver cancer detection.

However, this is just one example, and thus many analytes may be analyzed and analyte reaction results thereof may be various.

And also, an identification code means information including a separate code value required to obtain effective information about an analyte reaction result.

For example, the identification code may be manufacturing lot information about the analyte kit 300. The manufacturing lot information about the analyte kit 300 may be used to calibrate a result of reading an analyte reaction result.

As another example, the identification code may be an expire date of the analyte kit 300. The identification code including the expire date is used to determine whether the analyte kit is to be used.

The identification code may be implemented in various forms, such as bar code, color pattern, number, and character.

The analyte kit 300 having an analyte reaction result and an identification code in one side thereof is photographed using a camera 310. An image photographed by the camera 310 is analyzed by a processor 320 and then the analysis result is output to an output unit 330.

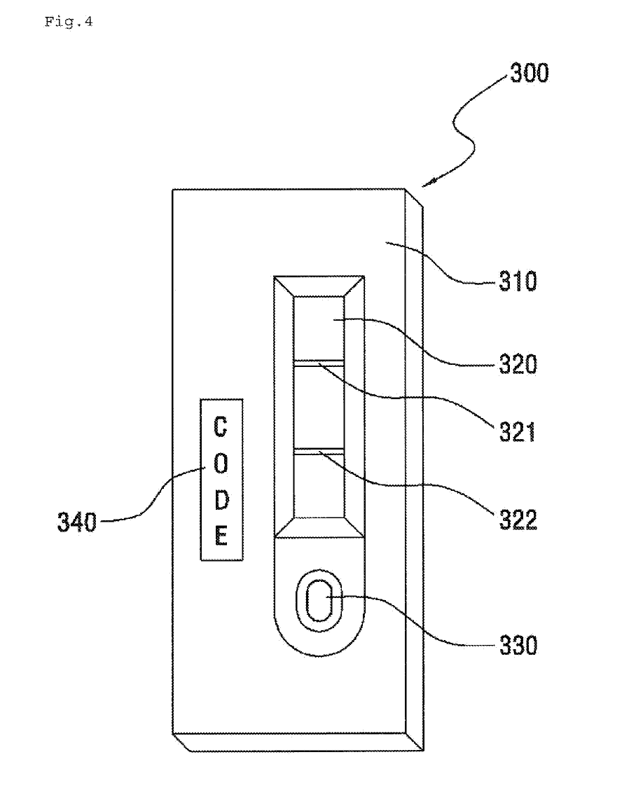

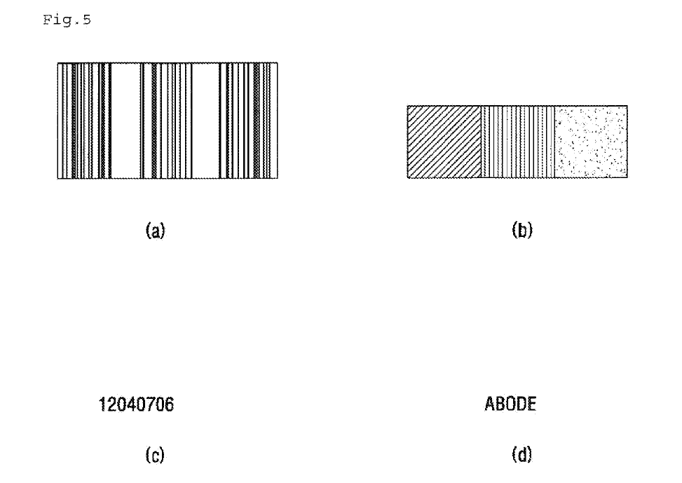

FIG. 4 is a view illustrating a first embodiment of an analyte kit according to the present invention.

As illustrated in FIG. 4, the analyte kit 300 includes an injection hole 330 for injecting an analyte, a display window 320 display an analysis result of the injected analyte, and an identification code 340.

In this case, the display window 320 indicates an reaction area where an analyte is reacted.

To obtain an analyte analysis result using a camera, the display window 320, reaction area, and identification code should be in the same side.

However since the camera reads the analyte reaction result and identification code separately, the display window 320 and the identification code 340 may be spaced apart by a certain interval.

And also, the identification code 340 exists in an area of the side of the analyte kit 300, which can be photographed by a camera.

The identification code 340 may be implemented in various methods, as shown in FIG. 5.

For example, it may be implemented with a barcode such as 1-dimensional barcode or 2-dimensional barcode, color pattern, numbers, or characters.

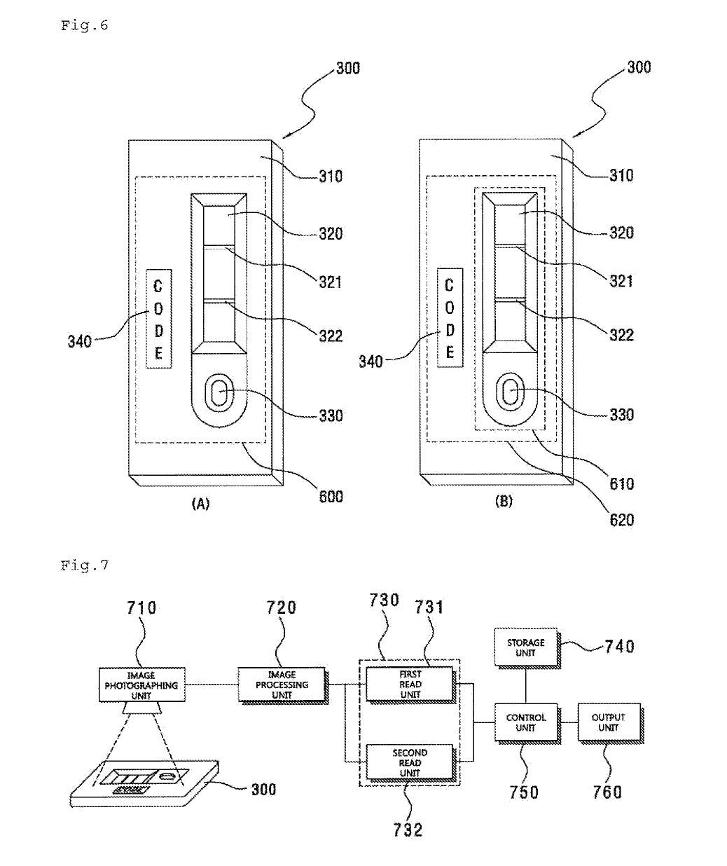

FIG. 6 is a view illustrating an analyte kit according to a second embodiment of the present invention.

As illustrated in FIG. 6(A), a virtual area 600 is shown, which can be recognized by a camera in the analyte kit 300 having the identification code 340 marked thereon.

Accordingly, the identification code 340 should be marked in the virtual area 600. Herein, the virtual area 600 may not be indicated. If necessary, the virtual area 600 may be indicated with a dotted line.

However, the virtual area 600 may be recognized in various methods by an algorithm during a process of reading an analyte reaction result or identification code.

In FIG. 6(B), a first virtual area 610 where an analyte reaction result of a display window 320 is photographed and a second virtual area 620 where an identification code is photographed are separated.

In this case, the analyte reaction result may be read from the first virtual area 610 of a photographed image, and the identification code may be read from the second virtual area 620 thereof. The second virtual area 620 may be a remaining area in the photographed image, except the first virtual area 610.

Herein, the first virtual area 610 and the second virtual area 620 may be recognized during a process by an algorithm even when there are no indications. And also, the areas may be indicated in a specific method.

Like this, the process of reading the analyte reaction result may be different from the process of reading the code information according to an area division method of the analyte kit 300.

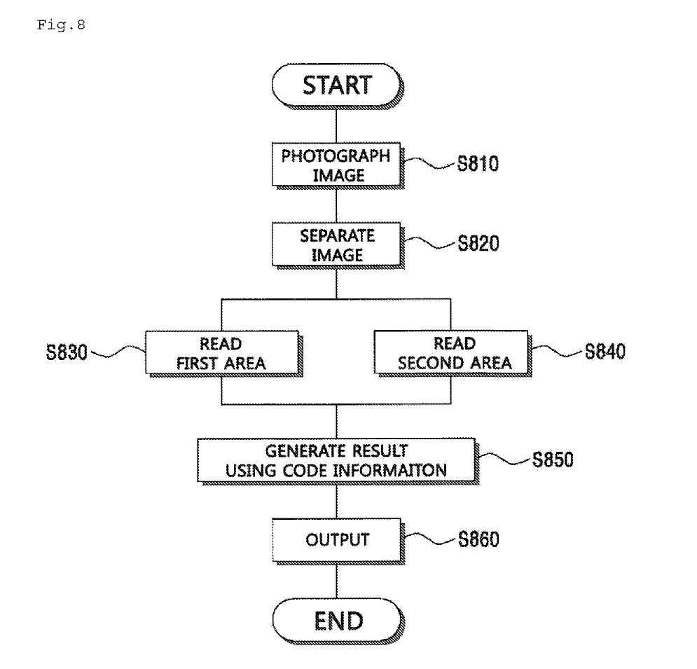

FIG. 7 is a view illustrating a configuration of a device for quantitatively measuring an analyte by using a camera according to the present invention.

As illustrated in FIG. 7, the quantitative measurement device includes an image photographing unit 710, an image processing unit 720, a read unit 730, a storage unit 740, a control unit 750, and a output unit 760. Herein, the image photographing unit 710 may be generally a camera.

The image photographing unit 710 photographs one side of the analyte kit 300 having an analyte reaction result and an identification code therein. In this case, the side of the analyte kit 300 is photographed at one time.

The image processing unit 720 separates an analyte reaction result image and an identification code image from the image of the side of the analyte kit 300 which is photographed by the image photographing unit 710.

The method of separating the analyte reaction result image and the identification code image may be implemented in various algorithms.

For example, the analyte reaction result image and the identification code image are separated according to their positions in a virtual area, as illustrated in FIG. 6(A). In a photographed image, a central portion may be recognized as the analyte reaction result image, and relatively an edge area may be recognized as the identification code area.

As another example, in a case where the analyte kit 300 has the first virtual area 610 and the second virtual area 620 as illustrated in FIG. 6(B), the first virtual area 610 is recognized as the analyte reaction result area, and the second virtual area 620 is recognized as the identification code area. The first virtual area 610 and the second virtual area 620 may be predetermined in the image processing unit 720.

The read unit 730 reads the analyte reaction result image and the identification code image, respectively.

The read unit 730 may have a first read unit 731 for reading the analyte reaction result and a second read unit 732 for reading the code information.

The control unit 750 allows the result of reading the analyte reaction result to be processed and then output by using the result of reading the identification code image. In this case, the control unit 750 may load information corresponding to the read identification code from the storage unit 740 storing information corresponding to identification codes and thus obtain effective information about the analyte.

In particular, when the identification code is manufacturing lot information about the analyte kit, the control unit 750 allows a result of reading the analyte reaction result to be calibrated using the manufacturing lot information which is read from the identification code.

When the identification code is an expire date, the control unit 750 allows an error message to be output through the output unit 760 when the expire date has passed according to a result of reading the identification code photographed by the image photographing unit 710.

And also, in a case where the identification code indicates a type of an analyte to be analyzed in an analyte kit, the control unit 750 allows an error message “unable to be analyzed” to be output through the output unit 760 when the analyte has the type which cannot be analyzed by the quantitative measurement device.

The output unit 760 outputs an effective end result for an analyte which is obtained by the control unit 750. Depending on the case, the output unit 760 may be referred to as various terms such as a display unit.

As another method, the image photographing unit 710 photographs one side of the analyte kit having an analyte reaction result and an identification code therein in the order of a first image and a second image.

In this case, the first image and the second image are the same or different with respect to a predetermined area.

The first image may be used for the analyte reaction result, and the second image may be used for the identification code.

The read unit 730 reads the analyte reaction result and the identification code from the first image and the second image, respectively, which are photographed by the image photographing unit 710.

In this case, the read unit 730 may extract and read the analyte reaction result and the identification code from the predetermined first image and second image, respectively.

The control unit 750 allows the result of reading the analyte reaction result to be processed and then output by using the result of reading the identification code image. In this case, the control unit 750 may load information corresponding to the read identification code from the storage unit 740 storing information corresponding to identification codes and thus obtain effective information about the analyte.

The output unit 760 outputs an effective end result for an analyte which is obtained by the control unit 750.

FIG. 8 is a view illustrating a method of quantitatively measuring an analyte by using a camera according to a first embodiment of the present invention.

As illustrated in FIG. 8, in the method of quantitatively measuring an analyte by using a camera according to a first embodiment of the present invention, one side of an analyte kit including an analyte reaction result and an identification code is photographed by an image photographing unit, i.e., a camera (S810).

After the side of the analyte kit is photographed, an analyte reaction result image included in a first area and an identification code image included in a second area are separated from the photographed image of the side of the analyte kit (S820). In this case, the first area and the second area may be predetermined.

After the analyte reaction result image and the identification code image are separated, the analyte reaction result of the first area is read (S830).

And also, the identification code of the second area is read (S840).

After the analyte reaction result and the identification code are read, a result of reading the analyte reaction result is analyzed using a result of reading the identification code, and then a reaction result is output (S850). In this case, analyzing the result of reading the analyte reaction result and then outputting the analysis result denotes loading information corresponding to the read identification code from a storage unit storing information corresponding to identification codes to obtain effective information about the analyte. After the effective information about the analyte is obtained, this information is output through an output unit (S860).

For example, in a case where the identification code includes manufacturing lot information about the analyte kit, a result of reading the analyte reaction result is calibrated using the manufacturing lot information, and a result of reading the calibrated analyte reaction result is output.

In a case where the identification code of the second area includes expire date information and is photographed by a camera before an analyte is injected, it is determined whether the expire date of the analyte kit included in the identification code has passed, and if the expire date has passed, an error message is output.

And also, in a case where the identification code includes analyte type information about types of analytes capable of being analyzed in the analyte kit and is photographed by a camera before the analyte is injected, if the analyte has the type which cannot be analyzed, an error message is output.

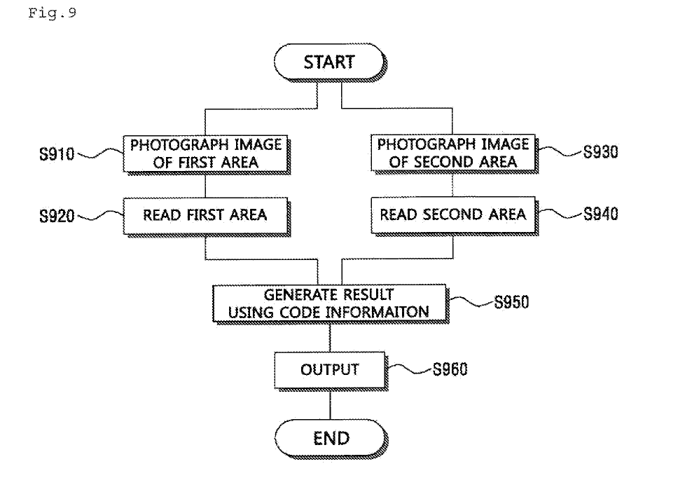

FIG. 9 is a view illustrating a method of quantitatively measuring an analyte by using a camera according to a second embodiment of the present invention.

As illustrated in FIG. 9, in the method of quantitatively measuring an analyte by using a camera according to a second embodiment of the present invention, one side of an analyte kit including an analyte reaction result and an identification code is photographed in the order of a first image and a second image (S910, S930). In this case, the first image denotes a first area image, and the second image denotes a second area image.

After the first image and the second image are photographed, the analyte reaction result is read from the first image (S920).

And also, the identification code is read from the second area (S940).

Accordingly, after the first image and the second image are photographed, a result of reading the analyte reaction result is analyzed using a result of reading of the identification code image, and then an analysis result is generated (S950).

In this case, in order to analyze the result of reading the analyte reaction result, data corresponding to the identification code stored in a storage unit may be used.

And then, the generated reaction result, i.e., effective information about the analyte is output (S960).

In particular, the cases where the identification code is the expire date information, the analyte type information, and the manufacturing lot information may be implemented through the processes of FIG. 8.

While the invention has been particularly shown and described with reference to exemplary embodiments thereof, it will be understood that various changes in form and details may be made therein without departing from the spirit and scope of the following claims.

1. A quantitative analyte measurement device for quantitatively measuring an analyte in blood, the quantitative analyte measurement device comprising:
a camera photographing a camera recognition area in one side of an analyte kit, wherein the camera recognition area comprises an analyte reaction result obtained by reaction of the analyte in blood and an identification code of the analyte kit; and
a read unit reading images of the photographed analyte reaction result and identification code.

2. The quantitative analyte measurement device of claim 1, further comprising a control unit allowing a result of reading the image of the analyte reaction result to be processed using a result of reading the image of the identification code.

3. The quantitative analyte measurement device of claim 1, wherein the camera photographs the analyte reaction result and the identification code at one time, and
the quantitative analyte measurement device further comprises an image processing unit separating the images of the analyte reaction result and identification code photographed by the camera from each other.

4. The quantitative analyte measurement device of claim 1, wherein the camera photographs the analyte reaction result and the identification code in the order of a first image and a second image, and
the read unit reads the analyte reaction result and the identification code from the first image and the second image, respectively.

5. The quantitative analyte measurement device of claim 1, wherein the identification code comprises at least one of a bar code, color pattern code, character, or number.

6. The quantitative analyte measurement device of claim 2, wherein the identification code comprises manufacturing lot information of the analyte kit, and
the control unit allows the result of reading the image of the analyte reaction result to be calibrated using the manufacturing lot information.

7. The quantitative analyte measurement device of claim 1, wherein the identification code comprises an expire date of the analyte kit,
the camera photographs the identification code before the analyte of blood is injected, and
the quantitative analyte measurement device further comprises a control unit allowing an error message to be output if the expire date of the analyte kit has passed.

8. The quantitative analyte measurement device of claim 1, wherein the identification code comprises analyte type information about types of analytes capable of being analyzed in the analyte kit,
the camera photographs the identification code before the analyte of blood is injected, and
the quantitative analyte measurement device further comprises a control unit allowing an error message to be output if the analyte has the type which cannot be analyzed in the analyte kit.

9. The quantitative analyte measurement device of claim 4, wherein the read unit extracts and reads the analyte reaction result and the identification code from a first virtual area and a second virtual area, respectively, of the camera recognition area in one side of the analyte kit.

10. A quantitative analyte measurement method of quantitatively measuring an analyte in blood by using a camera, the quantitative analyte measurement method comprising:
photographing a camera recognition area in one side of an analyte kit by a camera wherein the camera recognition area comprises an analyte reaction result obtained by reaction of the analyte in blood and an identification code of the analyte kit; and
reading images of the photographed analyte reaction result and identification code, respectively.

11. The quantitative analyte measurement method of claim 10, further comprising processing a result of reading the image of the analyte reaction result by using a result of reading the image of the identification code.

12. The quantitative analyte measurement method of claim 10, wherein the photographing comprises photographing the analyte reaction result and the identification code at one time, and
the quantitative analyte measurement method further comprises separating the images of the photographed analyte reaction result and identification code from each other.

13. The quantitative analyte measurement method of claim 10, wherein the photographing comprises photographing the analyte reaction result and the identification code in the order of a first image and a second image, and
the reading comprises reading the analyte reaction result and the identification code from the first image and the second image, respectively.

14. The quantitative analyte measurement method of claim 11, wherein the identification code comprises manufacturing lot information of the analyte kit, and
the processing comprises calibrating the result of reading the image of the analyte reaction result by using the manufacturing lot information.

15. The quantitative analyte measurement method of claim 10, wherein the identification code comprises an expire date of the analyte kit, and
the quantitative analyte measurement method further comprises:
photographing the identification code before the analyte of blood is injected; and
outputting an error message if the expire date of the analyte kit has passed.

16. The quantitative analyte measurement method of claim 10, wherein the identification code comprises analyte type information about types of analytes capable of being analyzed in the analyte kit,
the quantitative analyte measurement method further comprises: photographing the identification code by the camera before the analyte of blood is injected; and
outputting an error message if the analyte has the type which cannot be analyzed in the analyte kit.

17. An analyte kit used in a quantitative analyte measurement device for quantitatively measuring an analyte in blood by using a camera, the analyte kit comprises:
a display window displaying an analyte reaction result obtained by reaction of the analyte in blood; and
an identification code of the analyte kit,
wherein the display window and the identification code are formed in a camera recognition area in one side of the analyte kit.

18. The analyte kit of claim 17, wherein the camera recognition area comprises a first virtual area and a second virtual area, the first virtual area comprising the display window and the second virtual area comprising the identification code.

19. The analyte kit of claim 17, wherein the identification code comprises at least one of manufacturing lot information, an expire date, and analyte type information about types of analytes capable of being analyzed.

20. A quantitative analyte measurement system of quantitatively measuring an analyte in blood, the quantitative measurement system comprising:
the quantitative analyte measurement device according to claim 1; and
the analyte kit according to claim 17.
